(19) United States
(12) Patent Application Publication
(10) Pub. No.: US 2011/0152925 A1
(43) Pub. Date: Jun. 23, 2011

(54) Skin Preparation That Immobilizes Bacteria

(76) Inventors: Phillip A. Schorr, Cumming, GA (US); Douglas R. Hoffman, Greenville, WI (US); Ilona F. Weart, Roswell, GA (US); Kaiyuan Yang, Cumming, GA (US)

(21) Appl. No.: 12/644,642

(22) Filed: Dec. 22, 2009

(51) Int. Cl. A61B 17/03

(52) U.S. Cl. 606/214

(57) ABSTRACT

Skin sealants are usually applied over skin preps to seal the skin and hold any remaining bacteria in place prior to surgical incisions. This sealant is generally left on the skin after surgery. This two step process is time consuming, as each layer must dry before the next one is applied. A skin preparation is provided that immobilizes at least 95 percent of bacteria on the skin and is essentially free of cyanoacrylate. The skin preparation has a fugitive solvent and may also have antimicrobial compounds like iodophors, biguanides, phenols, quaternary amines, antibiotics and metals. This skin preparation may be used without an additional skin sealant or incise applied over it prior to surgery.

### BACKGROUND OF THE INVENTION

Surgical site infections (SSI) represent a significant source of patient morbidity and mortality in the United States and abroad. More than 30 million surgeries are performed each year in Europe and of these, as many as 2% or 600,000 patients will develop an infection of the surgical site. In the United States surgical site infections are the second most common hospital acquired infection. Of 40 million operations performed in the United States annually, 0.8 million to 2 million are associated with surgical site infections. SSIs prolong hospital stays by an average of 7.5 days and cost the nation between $130 million to $845 million each year. It is estimated that 40 percent to 60 percent of surgical site infections could be prevented.

Hospitals can incur significant added costs due to the occurrence of SSI, it is not unusual for SSI associated with cardiothoracic procedures to cost a hospital upwards of $250,000 per incident. Of course these costs are, in the end, borne by society in the form of added overall health care costs. Estimates of these societal costs range from 1 to 10 billion dollars annually.

New US government policies like the Centers for Medicare and Medicaid Services (CMS) Pay for Performance (P4P) plan are requiring hospitals to assume responsibility for avoidable nosocomial infections. Under this plan, if a patient acquires a condition during a hospital stay, Medicare will no longer pay the additional cost of the hospitalization. This approach encourages hospitals to take measures to prevent events from happening. Among the list of conditions for which additional payments would not be made include select SSI's. Plans such as P4P have brought a heightened awareness in hospitals and even in state legislatures, with hospitals taking greater action to implement new quality control process and state legislators moving on infection reporting.

Standards that are geared toward infection rate reduction include hand hygiene, aseptic technique, surgical draping, and creating/maintaining sterile field. In addition, a variety of additional approaches to reduce the microbial content of the skin around the incision site and prior to incision have been introduced to the market. Many antimicrobial treatments, specifically surgical skin preparation (prep) solutions, have been developed to accomplish this goal. Examples include formulations that contain alcohol, chlorhexidine gluconate, and iodorphors. While these prep solutions are able to significantly reduce the numbers of microbes present on the skin, it has long been realized that complete eradication of skin bacteria is not possible.

Standard surgical skin preps such as BETADINE® (Purdue Pharma) or CHLORAPREP® (Cardinal Healthcare) prep provide antimicrobial action when applied to the skin but they cannot completely sterilize the site. They also do not provide any level of bacterial immobilization. As a result, any remaining viable bacteria are able to migrate into the incision via irrigation fluids, bodily fluids, or manipulation. U.S. Pat. No. 5,916,882, believed to pertain to BETADINE®, includes alcohol, iodine and a preferably water soluble gel.

Existing film-forming preps such as DURAPREP® (3M) or PREVAIL® FX (Cardinal Healthcare) provide little or no bacterial immobilization (<90% of viable organisms immobilized). The adhesive strength of these films on skin is also relatively weak, which can lead to their unwanted and unanticipated removal during manipulation of the surgical site. U.S. Pat. No. 4,584,192, believed to pertain to DURAPREP®, teaches a film forming composition containing iodine and teaches away from the use of acid functional monomers (col. 5, line 55-58).

Other attempts have been made to supply film-forming compositions for surgical use. For example, U.S. Pat. No. 5,547,662 discloses a film forming skin prep that includes an anti-microbial agent and a colorant. This patent does not discuss the immobilization of bacteria. U.S. Pat. No. 6,139,856 provides a fluoride based film and includes an anti-microbial compound. WO 01/01994 discloses an aqueous solution for a film forming composition.

Since it is impossible to sterilize human skin prior to surgery, other approaches to limit microbial contamination of surgical incisions have been adapted to be used in conjunction with surgical preps. One such approach is based on immobilization of bacteria on the skin. These products work by reducing the likelihood that microbes surviving the prepping step migrate from the skin into the incision. Examples of such products include surgical incise drapes manufactured by 3M under the tradenames IOBAN® and STERIDRAPE®. Incise drapes are large sheets of adhesive film applied pre-operatively over the intended surgical incision site. Once applied, the surgeon makes the incision through the drape. The drape is then left in place until such time that the incision is closed, and the drape is partially or totally removed from the skin to allow wound closure.

In principal these film based incise drapes should afford benefits towards reducing wound contamination and thus ostensibly reducing the rate of SSI. In practice, however, various studies suggest that the effectiveness of these products is equivocal. Some studies have shown benefits of incise drape use for reducing wound contamination. Other studies have suggested that the product suffers from certain shortfalls including the loss of adhesion to wound margins with a subsequent failure in performance. Failure of conventional incise drapes to adhere to the margins of a skin incision significantly increases the SSI infection rate relative to surgeries in which the drapes do not experience such a failure.

Incise drapes and skin sealants are viewed as ancillary products to be used in conjunction with surgical skin preps, not as replacements. The health care worker is therefore required to keep two different products on hand and take the time to apply each to the patient prior to commencing the surgical procedure. Space and time are both very valuable commodities in the hospital and the operating room (OR) in particular. A single product that could both disinfect and immobilize bacteria on skin in a single step would provide a space and time saving advantage. It is clear that there exists a need for a skin prep or sealant that immobilizes bacteria and that may have biocidal activity.

### SUMMARY OF THE INVENTION

In response to the foregoing difficulties encountered by those of skill in the art, we have discovered an alcohol soluble, water insoluble film-forming composition having good adhesion to the skin and which immobilizes bacteria. This film-forming skin preparation composition may be used alone, i.e., without a skin sealant or incise applied over it prior to surgery.

The composition has a fugitive solvent and a compound that may be an acrylic acid ester of an alkyl alcohol and a methacrylic acid ester of an alkyl alcohol and acrylic or methacrylic acid units, an alkyl vinyl ether and an acid ester of maleic anhydride units or a methacrylic acid ester of an alkyl alcohol and vinyl pyridine units and an alkyl acrylamide and acrylic or methacrylic acid units and a natural gum resin. The formulation may optionally contain a secondary antimicrobial agent that may provide extended biocidal activity.

### DETAILED DESCRIPTION OF THE INVENTION

This disclosure concerns skin friendly, film-forming compositions that can effectively immobilize and optionally kill skin flora in a single application step.

The formulation desirably should contain a fugitive solvent, desirably alcohol, to ensure rapid drying and optional antimicrobial efficacy during the application step and be cyanoacrylate free. The resulting film that forms on the skin should:
Be water-resistant to prevent wash-off during irrigation or contact with bodily fluids;
Possess good skin adhesion and flexibility to remain durably intact during manipulation that occurs throughout a surgical procedure;
Be able to immobilize at least 90% and preferably greater than 99% of viable bacteria that are present on the skin under the applied film.

The formulation may optionally contain a secondary antimicrobial agent that may provide extended biocidal activity. Examples of such agents include iodophors (PVP-I), biguanides (CHG), phenols (PCMX), quaternary amines (benzalkonium chloride), antibiotics, oxidants and metals (silver). The film may also contain a tackifying agent (natural gums, adhesives, etc) to improve the attachment of any secondary drapes or dressings and a colorant to indicate where it has been applied. Suitable gums include mastic gum, boswellin, benzoin compounds, and Elemi gum.

A fugitive solvent is a chemical that is volatile at temperatures between about 25° C. and about 40° C., i.e. about room temperature and above. Fugitive solvents include alcohols, esters, chlorinated hydrocarbons, esters, and chlorofluorocarbons. Exemplary fugitive solvents include isopropanol, ethanol, ethyl acetate, trichloroethane, and acetone.

Test procedures were developed to determine the candidates that best met the above criteria. These procedures are reported below.

### Immobilization Test Method

### Reagents

### Citrate Buffer

Prepare a 0.1 M solution of citric acid (21.01 g anhydrous reagent grade citric acid {HOC(COOH)(CH2COOH)2} in 1000 ml). Filter, sterilize and store at 4° C. (expires after 3 months). Prepare a 0.1 M solution of sodium citrate (29.41 g Na3C6H5O7.2H2O in 1000 mL). Filter, sterilize and store at 2-8° C. (expires after 3 months). CB: Measure 16.0 mL 0.1 M citric acid in a graduated cylinder. Add 34.0 mL sodium citrate to the cylinder. Bring to 100 mL with distilled water. Adjust pH to 5.5+/−0.1 with 1M NaOH and/or 5M HCl. Filter, sterilize and store at 2-8° C. (expires after 1 month).

### Phosphate Buffered Skin Eluant (PBSE)

Prepare a 10× stock solution of phosphate buffered saline (PBS) by dissolving 12.36 g Na2HPO4 (anhydrous reagent grade), 1.80 g NaH₂PO4.H2O (reagent grade) and 85.00 g NaCl (reagent grade) in 1000 mL. Filter, sterilize and store at room temperature for a period not to exceed 1 year. PBSE: Add 100 mL of 10×PBS stock solution to 850 ml of distilled water. Add 10.0 ml TRITON X-100, 20.0 ml of Tween-80, 1.00 g peptone, and 16.66 g lecithin to the PBS. Bring to a boil with constant mixing, remove from heat and continue mixing until lecithin is completely dissolved. Dispense into appropriate sized bottles and sterilize by autoclaving. Remove from autoclave and allow to cool slightly. Tighten caps and shake vigorously. Store at 2-8° C. Allow to acclimate to room temperature prior to use. Expires after 1 month.

NOTE: Sodium thiosulfate is to be added to the PBSE to achieve a final concentration of 0.1% (w/v) in the final solution. Addition of this constituent should be performed using aseptic techniques.

### Skin Samples

An appropriate number of 5 cm×5 cm skin samples were cut from an appropriate skin surrogate. In these studies, the skin surrogate used was non-sterile MEDISKIN® II skin surrogate (Brennen Medical, LLC, St. Paul, Minn., Catalog#I-188). MEDISKIN® II skin surrogate was stored at −20° C. and thawed under ambient conditions for 30-60 minutes. A minimum of 2 replicates were set up per code being investigated. The sampling area to be utilized in the cup scrub was demarcated on each skin sample using the stainless steel cup scrub ring and a surgical marking pen.

### Skin Inoculation Procedure

A 25 μL aliquot of the prepared inoculum (Bacillus stearothermophilus spores EM Science Catalog #1.11499.0001, ˜10⁷CFU/ml in sterile citrate buffer pH 5.5) was added to the center of each sample and spread evenly within the sampling area using a sterile glass rod. The sample was then allowed to stand with the Petri dish lids ajar until dry.

### Skin Treatment

The dried and inoculated skin samples were treated by pipetting 0.1 or 1.0 ml of the test film former substance (dosage of 0.004 and 0.04 ml/cm², respectively) to the center of the sample then spreading it evenly across the entire surface using a sterile glass rod. The skins were allowed to stand with the Petri dish lids ajar for at least 30 minutes, or until dry (maximum 120 minutes).

### Test System Recovery

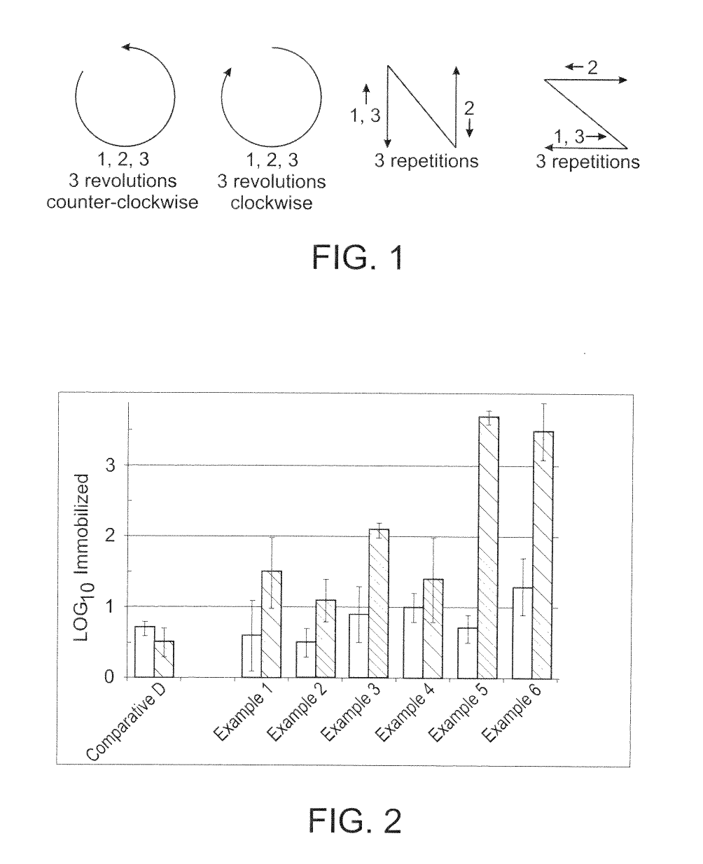

For each sample, a sterilized cup scrub ring was held firmly in place directly over the sampling area. While holding the ring in place, 2 mL of Phosphate Buffered Skin Eluent (PBSE) was delivered by pipette to the test site. Using a sterile glass rod, the test site was gently scraped following three repetitions of a counter clockwise, clockwise, up and down, and back and forth motion. See FIG. 1.

Using a sterile pipette, the eluent was removed from inside the cup scrub ring and transferred to a sterile tube. Following the first recovery process, an additional 2 mL of PBSE was added to the same test site, gently scraped using a sterile glass rod and transferred into the same pooled vessel and mixed. The vessel contained approximately 4 ml of eluent.

Serial 10-fold dilutions of the pooled vessels were made in PBSE. Each dilution was plated in duplicate on tryptic soy agar (TSA) to quantify the total number of viable spores recovered from each eluent sample.

### Incubation and Observation

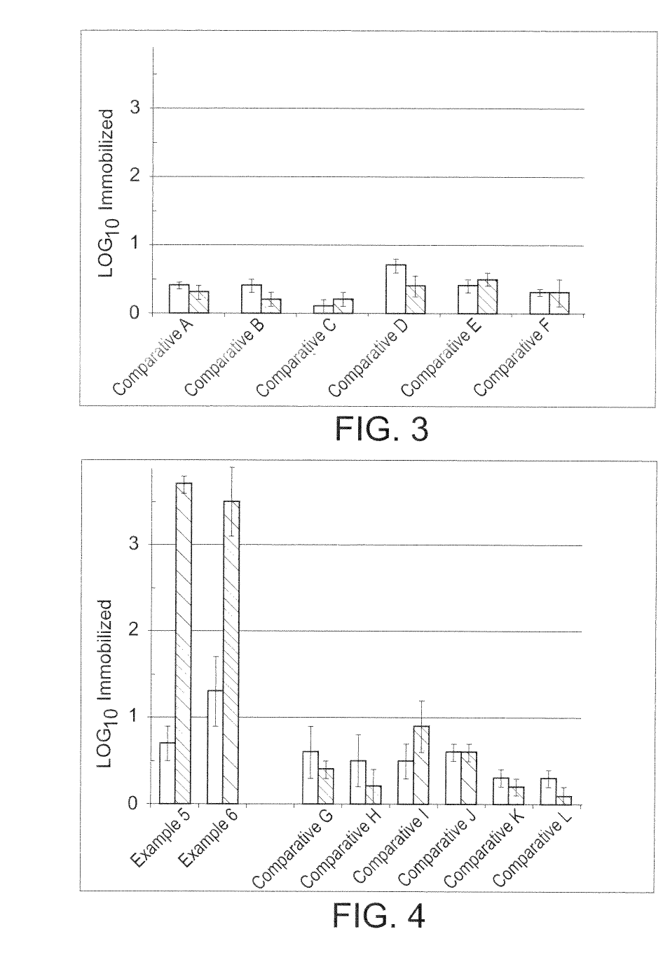

All TSA plates were incubated for 48±4 hours at 50±2° C. Following incubation, the total number of colony forming units (CFU) on each plate was enumerated and recorded. Using the plate counts, the total LOG₁₀ CFU/sample was calculated. Test codes containing a test substance treatment were compared against samples that were inoculated with spores but received no test substance treatment. The reduction in the number of CFUs due to the test substance is reported below under the column “immobilization”. I.e. LOG₁₀ untreated−LOG₁₀ treated=LOG₁₀ immobilization provided by the film-forming polymer. Results for the immobilization of exemplary materials and comparative materials are shown in FIGS. 2-4. The composition of the materials is described below.

### Adhesive Strength to Skin Test Method

The adhesive strength of films to the skin was determined using a variation of ASTM D5179-02 “Standard Test Method for Measuring Adhesion of Organic Coatings to Plastic Substrates by Direct Tensile Testing”. This method was chosen as it was found to be the most reproducible adhesive strength measurement on skin over other ASTM tests such as ASTM F-2256 and ASTM F2458. Additionally this method had the advantage of utilizing samples of the same size and polymer dosage level as the cup scrub method, thus allowing for straightforward comparison of adhesive strength to bacterial immobilization efficacy. All steps were completed in an environmentally-controlled room (22-24° C., 48-52% RH). A skin surrogate (Porcine Test Material, Cat#I-188 from Brennen Medical, LLC, St. Paul, Minn.) was cut to 5 cm×5 cm squares. The skin sample was then handled according to the following steps:
One skin sample was placed onto the center of a piece of double-sided tape (5 cm wide×˜15 cm long, 3M® 410B-7197-017). To ensure the skin sample was securely affixed to the tape, just prior to applying the skin sample the center of the double-side tape where the sample is placed was treated w/0.1-02 ml of cyanoacrylate glue. The glue was distributed in drops across the entire area where the sample was to be placed.
Each sample was treated with either 0.1 ml a cyanoacrylate composition or 0.1 ml of a film-forming polymer. The treatment was spread evenly across the sample surface using a glass rod and allowed to dry for a minimum of 15 minutes but no more than 40 minutes. In cases where the impact of surgical prep solutions on cyanoacrylate adhesion strength was being examined, 0.1 ml prep solution was applied and spread across the sample surface and allowed to dry a minimum of 15 minutes prior to application of the cyanoacrylate.
After the treatment dried, a sample was placed on the cohesion tester stand using the other side of the double-sided tape to which the sample was attached.
A test run was completed on the cohesion tester before each sample. This involved placing non-stick paper over the sample and running the cohesion tester for 3 seconds at 60 psi.
A piece of double-sided tape (2.5 cm wide×˜5 cm long, 3M® 4108-7197-017) was placed onto the bottom of a stainless steel stud with bottom surface dimensions of 2.5 cm×2.5 cm. The stud was placed on the cohesion tester with taped-side down. The total sampled surface was the same dimensions as the stud (2.5 cm×2.5 cm).
The test was then started and the stud and sample were brought into contact for 30 seconds at 60 psi. This was a sufficient amount of time for the tape on the stud to become well-adhered to the treatment film on the skin surface.
After 30 seconds, the stud and sample are pulled apart and the force necessary to pull them apart is quantified in kilograms of force. The stud was visually assessed to determine if the film was removed from the skin sample surface and ensure that the adhesion strength of the film to skin was truly measured. If the film was not removed from the sample, the test was not considered valid.

### Materials

Film-forming polymers were obtained from International Specialty Products (ISP), National Starch, and Sigma-Adrich and are listed below. The vendor number is included for the Sigma-Aldrich materials, since they are not sold under a specific trade name.

| Vendor | Film-Forming Polymers |
|---|---|
| International | Anatron-220-F |
| Specialty | Gantrez ES-435 |
| Products (ISP) | Gantrez SP-215 |
| | Gantrez ES-335 |
| | Styleze 2000 |
| National Starch | Dermacryl 79 |
| | Dermacryl C |
| Sigma-Aldrich | Mastic gum #286001 |
| | Poly(butyl methacrylate-co-methyl methacrylate) |
| | P(BMA-MMA) #445827 |
| | Poly(vinyl acetate-co-butyl maleate-co-isobornyl |
| | acrylate) P(VAc-BM-iBA) #434477 |
| | Poly(vinyl pyridine-co-butyl methacrylate) |
| | P(VP-BMA) #306258 |

### Exemplary Film Formers

Dermacryl 79 and Dermacryl C. The Dermacryl polymer series is commercially available from National Starch. Dermacryl 79 is a copolymer compound containing alkyl acrylamide and acrylic or methacrylic acid repeating units (see image A below) and is supplied as a white powder. Dermacryl C is a copolymer compound containing acrylic acid ester of an alkyl alcohol, methacrylic acid ester of an alkyl alcohol, and acrylic or methacrylic acid repeating units (see image B below) and is supplied as an aqueous emulsion. Both are designed to form clear, substantive, flexible films which can improve resistance to wear, enhance water resistance, and give a soft natural feel. They are commercially used as a waterproofing aid in mascara and other color cosmetics as well as sunscreens.

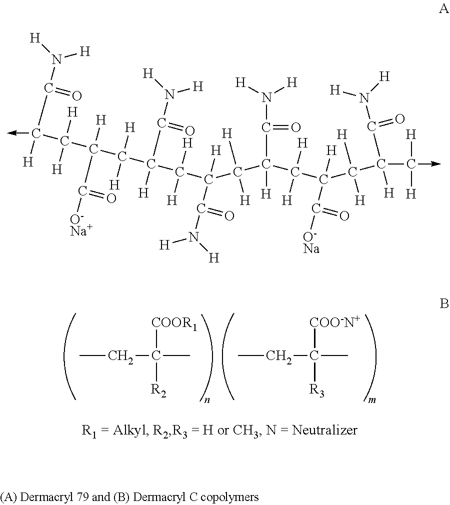

Gantrez ES-435. The Gantrez polymer family consists of copolymers of alkyl vinyl ether and maleic acid esters. They are designed to form a tough, clear film that is tack-free, has excellent substantivity, hair-holding properties and moisture resistance. They are commercially used in hair fixitive products, like hair sprays and gels. The individual versions of Gantrez cover a range of different alkyl ester functionalities and molecular weights. This diverse group of polymers gives the formulator a number of options to fine tune film adhesion strength and elasticity. For bacterial immobilization, it was found that the butyl ester version provides superior performance (Gantrez ES-435, image below). Gantrez EX-435 is a copolymer compound containing alkyl vinyl ether and acid ester of maleic anhydride repeating units (alkyl ester length n=4). Samples were supplied by ISP (International Specialty Products) as a viscous isopropyl alcohol solution with polymer loading at ˜50%.

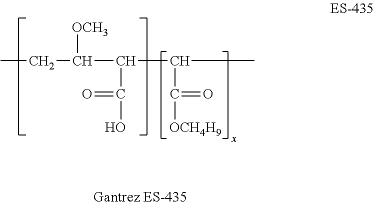

Acrylate copolymers. A wide range of acrylate-based copolymers were investigated as part of this study. These materials were chosen because they form excellent films and can easily be modified to obtain the desired solubility profile. The addition of co-monomers to the acrylate base monomer brings added benefits, like improved adhesion, elasticity, water resistance, etc. It was determined that vinylpyridine-co-butyl methacrylate copolymers provided superior bacteria immobilization compared to other acrylate copolymers. The chemical structure of this copolymer can be found below. Poly (vinylpyridine-co-butyl methacrylate) also referred to as P(VP-BMA) is a copolymer compound containing methacrylic acid ester of an alkyl alcohol and vinyl pyridine repeating units.

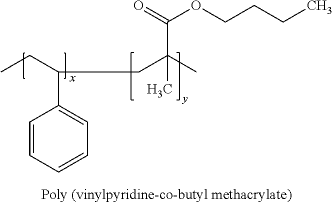

Mastic Gum. Mastic Gum is an example of a natural gum resin. It is produced by the Pistacia lentiscus tree (an evergreen shrub from the pistachio tree family). It has been used for a variety of gastric ailments in Mediterranean and Mideast countries for at least 3,000 years. In ancient times, mastic gum was highly revered for its medicinal properties in the relief of dyspepsia and other intestinal disorders. Other suitable gums include boswellin, benzoin compounds, and Elemi gum.

### Comparative Example Skin Preparations

Commercial skin preps. A series of commercially available skin preparations were obtained for testing as comparative examples. These preps tested are listed below in Table 1. These skin preps represent a wide range of compositions containing various active antimicrobial agents. Four of them contain an undisclosed film-forming agent in addition to the antimicrobial actives. All prep solutions were removed from their applicators (if applicable) and tested using the protocols described above. These comparative examples illustrate that existing commercial skin preps provide only minimal bacterial immobilization.

TABLE 1

| Commercial | | | Film |
|---|---|---|---|
| Product | Actives | Manufacturer | Former |
| Betadine ® | 1.0% Available Iodine | Purdue Pharma | No |
| ChloraPrep ® | 2% Chlorhexidine Gluconate | Cardinal | No |
| | 70% Isopropyl Alcohol | Health | |
| ACTIPREP ® | Zinc Pyrithione | Healthpoint | Yes |
| | (proprietary amount) | | |
| | 73% Ethyl Alcohol | | |
| Duraprep ™ | 0.70% Available Iodine | 3M | Yes |
| | 74% Isopropyl Alcohol | | |
| Prevail-FX ® | 0.83% Available Iodine | Cardinal | Yes |
| | 72.5% Isopropyl Alcohol | Health | |
| ExCel AP ® | 0.75% Available Iodine | Aplicare | Yes |
| | 72% Isopropyl Alcohol | | |

### Other Comparative Polymers.

Gantrez polymer series. Gantrez SP-215 and ES-335 are ethyl vinyl ether and maleic acid esters (see image below). Gantrez SP-215 is a copolymer compound containing alkyl vinyl ether and acid ester of maleic anhydride repeat units (alkyl ester length n=2). Gantrez ES-335 is a copolymer compound containing alkyl vinyl ether and acid ester of maleic anhydride repeat units (alkyl ester length n=3). Gantrez polymers were supplied by ISP (International Specialty Products) as viscous alcohol solutions with polymer loading at ˜50%. They are designed to form a tough, clear film that is tack-free, has excellent substantivity, hair-holding properties and moisture resistance. They are commercially used in hair fixitive products, like hair sprays and gels. These comparative examples demonstrate that selection of the maleic acid ester is important in achieving bacteria immobilization.

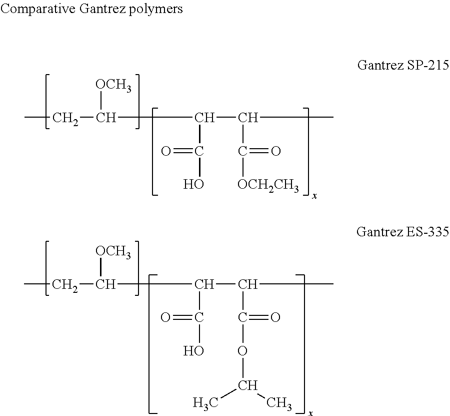

Styleze polymer series. Styleze is a copolymer compound containing vinyl-pyrrolidone, acrylic acid, and methacrylic acid ester of an alkyl alcohol repeating units (see image below). It is commercially available from ISP as a white powder. The Styleze 2000 product is designed to work in hair fixative products to provide improved moisture resistance and enhance substantivity to hair. This comparative example demonstrates that not all fixative agents from the cosmetic field provide bacteria immobilization.

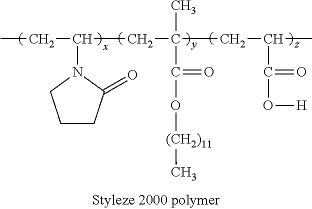

Antaron polymer series. These materials are copolymers of vinylpyrrolidone and long chain olefins. Anatron 220-F is a polymer compound containing alkyl substituted vinyl pyrrolidone units (see image below). Their solubility profile and viscosity in solution are controlled by the molecular structure and weight. Antaron V/WP grades are excellent film formers and provide water and wear resistance and moisture barrier properties. They are also very substantive to skin. This comparative example demonstrates that not all polymer films designed as water and/or wear resistant materials provide adequate bacteria immobilization.

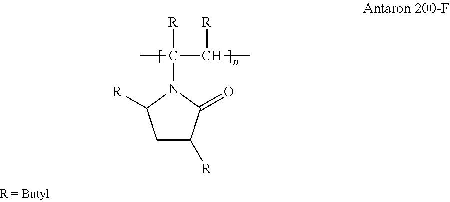

Acrylate copolymers. A wide range of acrylate-based copolymers were investigated as part of this study. These materials were chosen because they form excellent films and can be modified to obtain the desired solubility profile. The addition of co-monomers to the acrylate base monomer brings added benefits, like improved adhesion, elasticity, water resistance, etc. Some examples of the polymers investigated here and their chemical structures can be found in below. These comparative examples were selected to demonstrate that not all water-insoluble acrylic copolymers provide bacteria immobilization. Poly (butyl methacrylate-co-methyl methacrylate) also referred to as P(BMA-MMA) is a copolymer compound containing methacrylic acid esters of two distinct alkyl alcohols. Poly (vinyl acetate-co-butyl maleate-co-isobornyl acrylate) also referred to as P(VAc-BM-iBA) is a copolymer compound containing vinyl acetate, alkyl maleate, and acrylic acid ester of an alkyl alcohol repeating units.

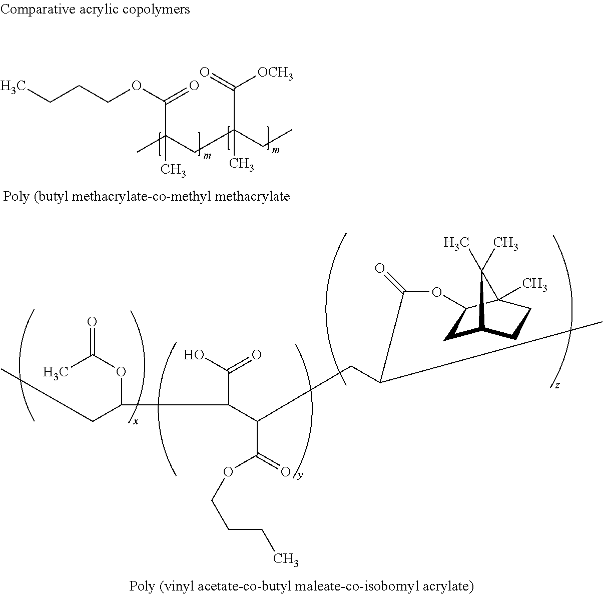

Materials Tested

| | Example compositions | |
|---|---|---|
| Example 1 | Mastic Gum | 30 g |
| | Ethanol | 70 g |
| Example 2 | Dermacryl 79 | 30 g |
| | Ethanol | 70 g |
| Example 3 | Dermacryl 79 | 15 g |
| | Mastic Gum | 15 g |
| | Ethanol | 70 g |
| Example 4 | P(VP-BMA) | 5 g |
| | Ethanol | 95 g |
| Example 5 | Gantrez-ES-435 (50 wt % IPA solution) | 50 g |
| | Isopropyl alcohol (IPA) | 50 g |
| Example 6 | Dermacryl C (40 wt % water emulsion) | 50 g |
| | Ethanol | 50 g |

| | Commercial Product |
|---|---|
| Comparative A | Betadine |
| Comparative B | ChloraPrep |
| Comparative C | ACTIPREP |
| Comparative D | Duraprep |
| Comparative E | Prevail-FX |
| Comparative F | ExCel AP |

Other comparative examples

| | | |
|---|---|---|
| Comparative G | Gantrez SP-215 (50% ethanol solution) | 50 g |
| | Ethanol | 50 g |
| Comparative H | Gantrez-ES-335 (50 wt % IPA solution) | 50 g |
| | Isopropyl alcohol (IPA) | 50 g |
| Comparative I | Styleze 2000 | 14 g |
| | Ethanol | 86 g |
| Comparative J | Anatron-220-F | 80 g |
| | Pentane | 20 g |
| Comparative K | P(VAc-BM-iBA) | 20 g |
| | Ethanol | 80 g |
| Comparative L | P(BMA-MMA) | 35 g |
| | Ethanol | 65 g |

Immobilization Results: Results are shown in tabular and graphical form for ease of comparison.

| | | | Immobilization | |
|---|---|---|---|---|
| | | | (LOG₁₀) | |
| | Example compositions | | 4 μL/cm² | 40 μL/cm² |
| Example 1 | Mastic Gum | 30 g | 0.6 | 1.5 |
| | Ethanol | 70 g | | |
| Example 2 | Dermacryl 79 | 30 g | 0.5 | 1.1 |
| | Ethanol | 70 g | | |
| Example 3 | Dermacryl 79 | 15 g | 0.9 | 2.5 |
| | Mastic Gum | 15 g | | |
| | Ethanol | 70 g | | |
| Example 4 | P(VP-BMA) | 5 g | 1.0 | 1.4 |
| | Ethanol | 95 g | | |
| Example 5 | Gantrez-ES-435 | 50 g | 0.7 | 3.7 |
| | (50 wt % IPA solution) | | | |
| | Isopropyl alcohol (IPA) | 50 g | | |
| Example 6 | Dermacryl C | 50 g | 1.3 | 3.5 |
| | (40 wt % water emulsion) | | | |
| | Ethanol | 50 g | | |

Immobilization
(LOG₁₀)

| | Commercial Product | 4 μL/cm² | 40 μL/cm² |
|---|---|---|---|
| Comparative A | Betadine | 0.4 | 0.3 |
| Comparative B | ChloraPrep | 0.4 | 0.2 |
| Comparative C | ACTIPREP | 0.1 | 0.2 |
| Comparative D | Duraprep | 0.7 | 0.4 |
| Comparative E | Prevail-FX | 0.4 | 0.5 |
| Comparative F | ExCel AP | 0.3 | 0.3 |

| | | | Immobilization | |
|---|---|---|---|---|
| | | | (LOG₁₀) | |
| | Other comparative examples | | 4 μL/cm² | 40 μL/cm² |
| Comparative G | Gantrez SP-215 | 50 g | 0.6 | 0.4 |
| | (50% ethanol solution) | | | |
| | Ethanol | 50 g | | |
| Comparative H | Gantrez-ES-335 | 50 g | 0.5 | 0.2 |
| | (50 wt % IPA solution) | | | |
| | Isopropyl alcohol (IPA) | 50 g | | |
| Comparative I | Styleze 2000 | 14 g | 0.5 | 0.9 |
| | Ethanol | 86 g | | |
| Comparative J | Anatron-220-F | 80 g | 0.6 | 0.6 |
| | Pentane | 20 g | | |
| Comparative K | P(VAc-BM-iBA) | 20 g | 0.3 | 0.2 |
| | Ethanol | 80 g | | |
| Comparative L | P(BMA-MMA) | 35 g | 0.3 | 0.0 |
| | Ethanol | 65 g | | |

It was found that the film forming compound should be present in an amount between 5 and 50 weight percent, more particularly between 10 and 30 weight percent, of the skin preparation formulation.

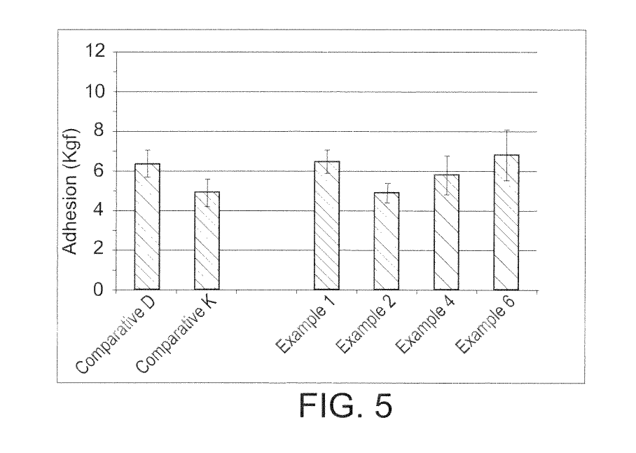

Adhesive Strength to Skin Test Results: Only a few of the examples and comparatives were tested as it was believed skin adhesion was adequate for most materials. The results are shown graphically in FIG. 5.

As can clearly be seen from the results, the exemplary materials had much better immobilization characteristics than the comparatives.

In addition to being used as a traditional skin preparation, i.e. as a film forming barrier through which a surgical incision is made, the indicator and curable preparation composition may also be used like a bandage to close and/or cover wounds, bruises, abrasions, burns, acne, blisters, bites, stings, nails, cuticles, punctures, cuts and other disruptions in the skin to protect them from subsequent contamination or indicate the presence due to growth of pre-contamination areas. The use of the skin preparation composition would therefore not be limited to medical personnel and would not require the use of a skin prep before the skin preparation is applied.

Wound protection is critical in permitting the healing process to take place. Traditional adhesive bandages and gauze wound dressings have been used by the consumer to treat/dress acute wounds or skin irritations. Such adhesive bandages are generally passive, in that they offer little or no chemical treatment for wound healing. Rather, they primarily serve to exert low levels of pressure on the wound, protect the wound from exposure to the environment, and absorb any exudates, which are produced from the wound site. Such bandages generally include a base layer, which is the layer seen by the consumer following application of the bandage to the wound. Such a layer is typically formed from a polymeric material such as a film, nonwoven web, or combination thereof, and may be perforated in some fashion to allow for flexibility and/or further breathability. This layer often includes a film component, having a top side surface which is seen by the consumer after application of the bandage to the wound site, and a bottom side surface (skin contacting surface). A skin-friendly adhesive is usually placed over the base layer bottom side surface to provide a means for attaching the bandage to the consumer. Alternatively, a separate adhesive tape is used to attach the bandage/wound dressing to the wound site, if the bandage/wound dressing is of the nonadhesive type. In the center of the base layer bottom side surface is traditionally positioned an absorbent pad for absorbing exudates from the wound. Finally, a non-stick perforated film layer is normally positioned over the absorbent pad layer, to provide a barrier between the absorbent pad and the wound itself. This allows the wound fluid to move through the perforated layer without sticking to the wound site. Typically the absorbent pad in such bandage does not include any medicinal components, although comparatively recently, bandage manufacturers have started including antibiotic agents on or within bandages to encourage wound healing.

The skin preparation of this invention can replace this seemingly complicated bandage construction with a single liquid treatment that will dry to a flexible preparation that protects a wound much like a bandage would. Additionally, medicaments such as antibiotic agents may be blended in effective amounts with the composition to provide additional benefits in the area of microbial inhibition and the promotion of wound healing. The preparation may be applied to provide an effectively thick preparation over the surface of the superficial wound, burn or abrasion. Because the to-be-treated wound is superficial and does not extend beyond the dermal layer, any polymeric residues diffusing into or forming in the wound will be naturally extruded from the skin. Generally, the preparation provides an adhesive film preparation over the wound area which when set is satisfactorily flexible and adherent to the tissue without premature peeling or cracking. The preparation generally has a thickness of less than about 0.5 millimeter (mm).

Sealant preparations of such thicknesses form a physical barrier layer over superficial wounds which provide protection for the wound in the same manner as a conventional bandage. Specifically, the preparation provides an almost airtight, waterproof seal around the wound which does not need to be replaced when the wound gets wet. Once applied, the preparation prevents bacterial and contaminant entry into the wound, thus reducing the rate of secondary infection. Generally, the adhesive preparation does not limit dexterity and promotes faster wound healing. Additionally, unlike conventional bandages, the preparation naturally sloughs off the skin within 2-3 days after application and, accordingly, avoids the discomfort associated with removal of conventional bandages from the skin. However, if early removal of this polymeric preparation is desired, such can be achieved by use of solvents such as acetone. Further discussion of this use may be found in U.S. Pat. No. 6,342,213.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A skin preparation for surgical use comprising a fugitive solvent and a compound selected from the group consisting of;
a) an acrylic acid ester of an alkyl alcohol and a methacrylic acid ester of an alkyl alcohol and acrylic or methacrylic acid units;
b) an alkyl vinyl ether and an acid ester of maleic anhydride units;
c) a methacrylic acid ester of an alkyl alcohol and vinyl pyridine units and;
d) an alkyl acrylamide and acrylic or methacrylic acid units with a natural gum resin.

2. The skin preparation of claim 1 wherein said fugitive solvent is selected from the group consisting of ethanol, isopropanol, ethyl acetate and acetone.

3. The skin preparation of claim 1 further comprising an antimicrobial compound selected from the group consisting of iodophors, biguanides, phenols, quaternary amines, antibiotics, oxidants and metals.

4. The skin preparation of claim 1 further comprising a tackifying agent that is a gum selected from the group consisting of mastic gum, boswellin, benzoin compounds, and Elemi gum.

5. The skin preparation of claim 1, which, when applied to the skin and allowed to dry, provides immobilization of at least 90 percent of bacteria present on said skin.

6. The preparation claim 1 wherein said immobilization is of at least 95 percent of said bacteria.

7. The preparation of claim 1 wherein said immobilization is of at least 99 percent of said bacteria.

8. The preparation of claim 1 wherein said compound is present in an amount between 5 and 50 weight percent.

9. The preparation of claim 1 wherein said compound is present in an amount between 10 and 30 weight percent.

10. The preparation of claim 1 wherein said preparation is essentially free of cyanoacrylate.

11. The preparation of claim 1 wherein no additional skin sealant or incise is applied over the preparation prior to surgery.

12. A skin preparation for surgical use comprising a fugitive solvent and a film-forming polymer wherein said preparation, when applied to the skin and allowed to dry, immobilizes 99 percent of bacteria present on said skin.

13. The skin preparation of claim 12 further comprising an antimicrobial compound selected from the group consisting of iodophors, biguanides, phenols, quaternary amines, antibiotics, oxidants and metals.
